(19) United States
(12) Patent Application Publication
(10) Pub. No.: US 2022/0287688 A1
(43) Pub. Date: Sep. 15, 2022

(54) ULTRASONIC DIAGNOSTIC APPARATUS, DETERMINATION METHOD, AND DETERMINATION PROGRAM

(71) Applicant: Konica Minolta, Inc., Tokyo (JP)

(72) Inventors: Tomohito SAKAI, Yokohama-shi (JP); Ryota TORII, Tokyo (JP); Jo SHIKAMA, Tokyo (JP)

(21) Appl. No.: 17/652,591

(22) Filed: Feb. 25, 2022

(30) Foreign Application Priority Data
Mar. 10, 2021 (JP) 2021-038289

(51) Int. Cl. A61B 8/00; A61B 8/08; G16H 50/20; G16H 30/40; G16H 40/63

(52) U.S. Cl. CPC A61B 8/54; A61B 8/469; A61B 8/461; A61B 8/5207; G16H 50/20; G16H 30/40; G16H 40/63; A61B 8/4488

(57) ABSTRACT

An ultrasonic diagnostic apparatus for generating an ultrasonic image of a subject in accordance with a set value of a plurality of scanning control parameters having a mutual relationship includes: a hardware processor that sets a setting range narrower than a limit range of the ultrasonic diagnostic apparatus, for at least one scanning control parameter among the plurality of scanning control parameters; and determines a set value of the plurality of scanning control parameters based on the setting range.

The entire disclosure of Japanese patent Application No. 2021-038289, filed on Mar. 10, 2021, is incorporated herein by reference in its entirety.

### BACKGROUND

### Technological Field

The present invention relates to an ultrasonic diagnostic apparatus, a determination method, and a determination program.

### Description of the Related Art

Conventionally, as one of medical image diagnostic apparatuses, there is known an ultrasonic diagnostic apparatus that visualizes a shape, a property, or dynamics inside a subject as an ultrasonic image by transmitting an ultrasonic wave toward the subject, receiving a reflected wave, and performing predetermined signal processing on a reception signal. Since the ultrasonic diagnostic apparatus can acquire an ultrasonic image by a simple operation of applying an ultrasonic probe to a body surface or inserting the ultrasonic probe into a body, the ultrasonic diagnostic apparatus is safe, and a burden on the subject is small.

In general, an ultrasonic diagnostic apparatus generates an ultrasonic image by setting values of a plurality of scanning control parameters such as a scanning range, a scanning line density, and a frame rate. The scanning range is a width of a diagnostic region scanned by an ultrasonic beam, or a depth of view. The scanning line density is the number of scanning lines (a center line of an ultrasonic beam) per unit area. The frame rate is the number of frames (ultrasonic images) per unit time.

Since these plurality of scanning control parameters have a relative relationship of trade-off, it is necessary to set a value of the scanning control parameter according to a purpose of inspection or the like.

For example, JP 2011-239906 A discloses an apparatus capable of stopping operation being executed and returning to a default condition in a case where it is desired to change one scanning control parameter during image capturing.

In addition, JP 2014-171541 A discloses an apparatus that controls a transmission/reception unit on the basis of a relative relationship between the number of times of ultrasonic transmission/reception and other scanning control parameters.

Further, JP S63-11137 A and JP S54-162881 A disclose apparatuses that change conditions of a scanning control parameter in accordance with an enlargement ratio of an ultrasonic image.

However, in the techniques described in JP 2011-239906 A, JP 2014-171541 A, JP S63-11137 A, and JP S54-162881 A, when a value of one scanning control parameter is set, other scanning control parameters are limited due to a relative relationship of trade-off. Therefore, in some cases, any of other scanning control parameters may be an inferior condition, and thus a value of the scanning control parameter has been unable to be flexibly set in the conventional technique.

### SUMMARY

An object of the present invention is to provide an ultrasonic diagnostic apparatus, a determination method, and a determination program capable of flexibly setting a set value of a scanning control parameter.

To achieve the abovementioned object, according to an aspect of the present invention, an ultrasonic diagnostic apparatus for generating an ultrasonic image of a subject in accordance with a set value of a plurality of scanning control parameters having a mutual relationship, reflecting one aspect of the present invention comprises a hardware processor that

sets a setting range narrower than a limit range of the ultrasonic diagnostic apparatus, for at least one scanning control parameter among the plurality of scanning control parameters; and

determines a set value of the plurality of scanning control parameters based on the setting range.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a view illustrating an appearance of an ultrasonic diagnostic apparatus according to an embodiment of the present invention;

FIG. 2 is a view illustrating an example of a scannable range of an ultrasonic probe;

FIG. 3 is a block diagram illustrating a main part of a control system of the ultrasonic diagnostic apparatus;

FIG. 4 is a graph illustrating a relationship between a plurality of scanning control parameters;

FIG. 5 is a graph for explaining an example of a determination method for a set value of a scanning control parameter;

FIG. 6 is a flowchart illustrating an example of an operation example of determination control on a scanning control parameter in a control part;

FIG. 7 is a graph for explaining an example of a determination method for a set value of a scanning control parameter;

FIG. 8 is a block diagram illustrating a main part of a control system of an ultrasonic diagnostic apparatus according to a modification;

FIG. 9 is a graph for explaining an example of a determination method for a set value of a scanning control parameter; and

FIG. 10 is a graph for explaining an example of a determination method for a set value of a scanning control parameter.

### DETAILED DESCRIPTION OF EMBODIMENTS

Hereinafter, one or more embodiments of the present invention will be described with reference to the drawings. However, the scope of the invention is not limited to the disclosed embodiments.

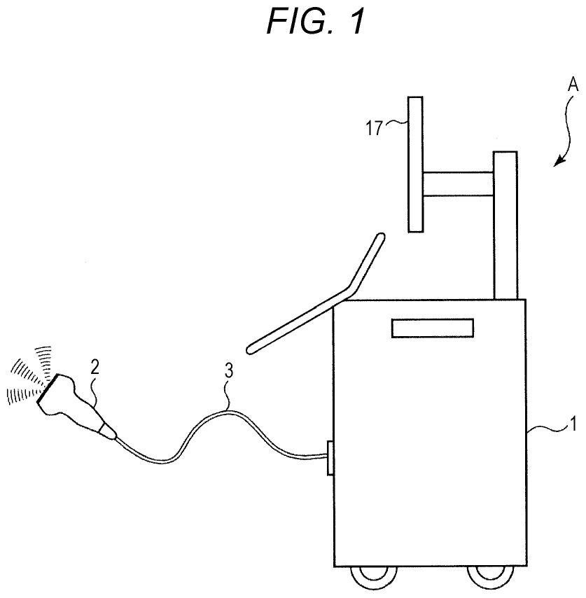

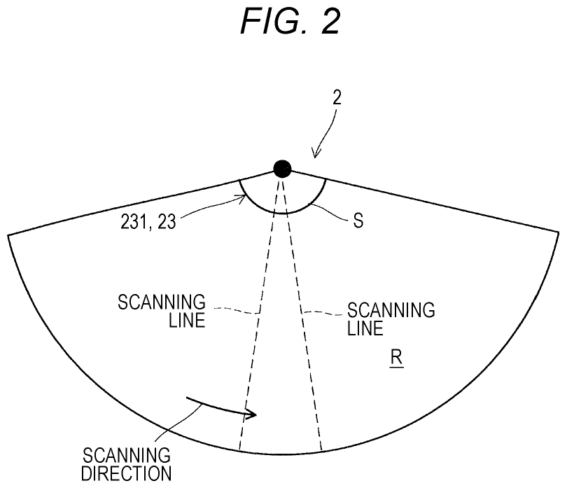

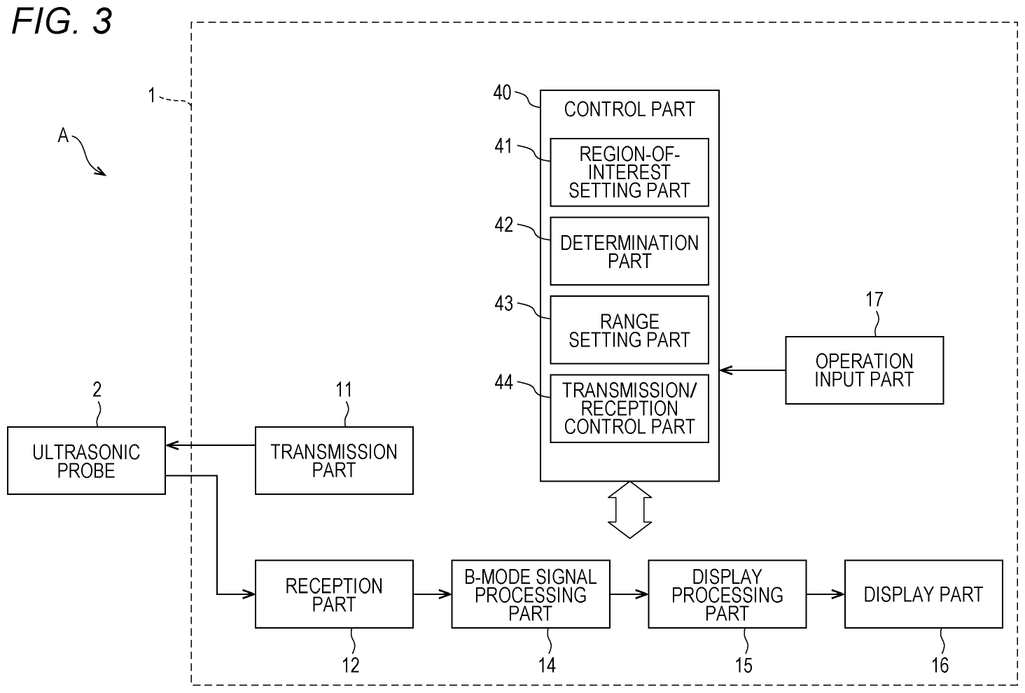

FIG. 1 is a view illustrating an appearance of an ultrasonic diagnostic apparatus A according to an embodiment of the present invention. FIG. 2 is a view illustrating an example of a scannable range of an ultrasonic probe 2. FIG. 3 is a block diagram illustrating a main part of a control system of the ultrasonic diagnostic apparatus A.

As illustrated in FIG. 1, the ultrasonic diagnostic apparatus A includes an ultrasonic diagnostic apparatus main body 1 and an ultrasonic probe 2. The ultrasonic diagnostic apparatus main body 1 and the ultrasonic probe 2 are connected via a cable 3. The ultrasonic probe 2 may be connected to the ultrasonic diagnostic apparatus main body 1 via wireless communication.

The ultrasonic diagnostic apparatus A is used to visualize a shape, a property, or dynamics inside a subject as an ultrasonic image and perform image diagnosis. The ultrasonic diagnostic apparatus A has a B mode for displaying a B-mode image alone, as a display mode. The ultrasonic diagnostic apparatus A may have a CFM mode in which a color flow mapping (CFM) image obtained by a color Doppler method is superimposed and displayed on a B-mode image.

The ultrasonic probe 2 transmits an ultrasonic wave to the subject, receives an ultrasonic echo reflected by the subject, converts the ultrasonic echo into a reception signal, and transmits the reception signal to the ultrasonic diagnostic apparatus main body 1. The ultrasonic probe 2 is a probe compatible with an electronic scanning method, and for example, a linear probe, a convex probe, or a sector probe can be applied. In the present embodiment, a case will be described in which an ultrasonic probe (for example, a convex probe) capable of handling a wider diagnostic region is applied as the ultrasonic probe 2.

As illustrated in FIG. 2, the ultrasonic probe 2 includes a transducer array 23. The transducer array 23 includes a plurality of transducers 231 arranged in a scanning direction.

The plurality of transducers 231 are arranged such that transducer surfaces S are arranged on an arc. Therefore, the scanning direction is a direction along the arc formed by the transducer surface S (for example, a counterclockwise direction in the figure). Such a transducer array 23 causes a diagnostic region R in the ultrasonic diagnostic apparatus A to be a fan-shaped region. In FIG. 2, each of the plurality of transducers 231 is indicated by a line connecting the plurality of transducers with a curve.

According to the ultrasonic probe 2, an ultrasonic wave can be made to converge in the scanning direction (so-called electronic focus) by sequentially switching the transducer 231 to be driven in the scanning direction.

Note that scanning lines corresponding to the number of transducers 231 pass through the diagnostic region R, but in FIG. 2, two scanning lines alone of ultrasonic waves are illustrated in consideration of visibility of the figure. Further, the two scanning lines illustrated in FIG. 2 correspond to two transducers 231 adjacent to each other in the scanning direction among the plurality of transducers 231.

The ultrasonic diagnostic apparatus main body 1 visualizes an internal state of a subject as an ultrasonic image, by using a reception signal from the ultrasonic probe 2. As illustrated in FIG. 3, the ultrasonic diagnostic apparatus main body 1 includes a transmission part 11, a reception part 12, a B-mode signal processing part 14, a display processing part 15, a display part 16, an operation input part 17, a control part 40, and the like.

The transmission part 11, the reception part 12, the B-mode signal processing part 14, and the display processing part 15 include, for example, at least one dedicated hardware (electronic circuit) corresponding to each process, such as a digital signal processor (DSP), an application specific integrated circuit (ASIC), or a programmable logic device (PLD).

The control part 40 includes a central processing unit (CPU) as a computation/control device, a read only memory (ROM) and a random access memory (RAM) as a main memory, and the like. The ROM stores a basic program and basic setting data. The CPU reads a program corresponding to a processing content from the ROM, expands the program in the RAM, and executes the expanded program to centrally control operation of each functional block (the transmission part 11, the reception part 12, the B-mode signal processing part 14, the display processing part 15, and the display part 16) of the ultrasonic diagnostic apparatus main body 1.

In the present embodiment, a function of each functional block is implemented by cooperation between each hardware included in the functional block and the control part 40. Note that a part or all of the function of each functional block may be implemented by the control part 40 executing a program, or each functional block may have a configuration capable of executing the program.

The control part 40 includes a region-of-interest setting part 41, a determination part 42, a range setting part 43, and a transmission/reception control part 44.

The region-of-interest setting part 41 sets a region of interest of an ultrasonic image. The region of interest is a partial region of the ultrasonic image, and is, for example, a region that is a portion where an observer (user) desires detailed observation in a diagnostic region of the ultrasonic diagnostic apparatus A.

The determination part 42 determines a set value of a plurality of scanning control parameters having a mutual relationship. The plurality of scanning control parameters are a scanning line density, a scanning range, and a frame rate. Note that the scanning control parameter may include parameters other than the scanning line density, the scanning range, and the frame rate.

The range setting part 43 sets a range of the scanning control parameter. Details of a determination method for a scanning control parameter by the determination part 42 and the range setting part 43 will be described later.

The transmission/reception control part 44 drives the ultrasonic probe 2 by controlling the transmission part 11, the reception part 12, and the like in accordance with a set value determined by the determination part 42. As a result, the ultrasonic diagnostic apparatus A generates an ultrasonic image of the subject.

The transmission part 11 generates a transmission signal (drive signal) in accordance with an instruction from the control part 40, and outputs the transmission signal to the ultrasonic probe 2. Specifically, the transmission part 11 controls driving of the ultrasonic probe 2 on the basis of a set value that is set by the control part 40. Although not illustrated, the transmission part 11 includes, for example, a clock generation circuit, a pulse generation circuit, a pulse width setting part, and a delay circuit.

The clock generation circuit generates a clock signal that determines a transmission timing and a transmission frequency of a pulse signal. The pulse generation circuit generates a bipolar rectangular wave pulse having a preset voltage amplitude at a predetermined period. The pulse width setting part sets a pulse width of a rectangular wave pulse outputted from the pulse generation circuit. The rectangular wave pulses generated by the pulse generation circuit are separated into different wiring paths for individual transducers 231 of the ultrasonic probe 2, before or after being inputted to the pulse width setting part. The delay circuit delays a generated rectangular wave pulse according to a drive timing of each of the transducers 231, and outputs the rectangular wave pulse to the ultrasonic probe 2.

By controlling the drive timing of the transducer 231, scanning line angles of a plurality of ultrasonic waves transmitted in one scanning can be made different.

In accordance with an instruction from the control part 40, the reception part 12 receives a reception signal from the ultrasonic probe 2 and outputs the reception signal to the B-mode signal processing part 14. Although not illustrated, the reception part 12 includes, for example, an amplifier, an A/D conversion circuit, and a phasing addition circuit.

The amplifier individually amplifies a reception signal corresponding to the ultrasonic wave received by each transducer 231 of the ultrasonic probe 2 at a predetermined amplification factor set in advance. The A/D conversion circuit converts the amplified reception signal into digital data at a predetermined sampling frequency. The phasing addition circuit applies a delay time to the A/D-converted reception signal for each wiring path corresponding to the transducer 231 to adjust a time phase, and adds the reception signals (phasing addition).

The B-mode signal processing part 14 generates B-mode image data by performing envelope detection processing, logarithmic compression processing, and the like on reception data for a B-mode image from the reception part 12 in accordance with an instruction from the control part 40, to adjust a dynamic range and a gain to perform luminance conversion.

In accordance with an instruction from the control part 40, the display processing part 15 converts image data generated by the B-mode signal processing part 14 into a display signal compatible with the display part 16 and outputs the display signal, and causes the display part 16 to display the B-mode image. Note that the display processing part 15 includes a digital scan converter (DSC) that performs coordinate conversion and pixel interpolation according to a type of the ultrasonic probe 2.

Further, when receiving a command to enlarge and display the region of interest from the operation input part 17 or the like, the display processing part 15 outputs a display signal to enlarge and display image data corresponding to the region of interest, to the display part 16.

The display part 16 includes, for example, a liquid crystal display, an organic EL display, a CRT display, or the like. The display part 16 displays an image on the basis of a display signal from the display processing part 15 in accordance with an instruction from the control part 40.

The operation input part 17 receives, for example, an input of information regarding diagnosis. The operation input part 17 includes, for example, an operation panel having a plurality of input switches, a keyboard, a mouse, and the like. The user can set a region of interest, a diagnosis site, a type of the ultrasonic probe 2, and the like via the operation input part 17.

Note that an external device (for example, a tablet terminal) communicably connected to the ultrasonic diagnostic apparatus main body 1 can also be applied to at least one of the display part 16 or the operation input part 17.

Next, a determination method for a scanning control parameter by the determination part 42 and the range setting part 43 will be described.

The range setting part 43 sets a setting range of one scanning control parameter among a plurality of scanning control parameters. The setting range is a range narrower than a limit range of the one scanning control parameter in the ultrasonic diagnostic apparatus A, and is set on the basis of, for example, a user's instruction, a diagnosis purpose of the subject, a specification of the ultrasonic diagnostic apparatus A, and the like. In the present embodiment, the range setting part 43 sets a setting range on the basis of a user's instruction. Further, an upper limit value and a lower limit value of the setting range are indicated by absolute values of a scanning control parameter.

Further, the limit range is a range from a maximum value to a minimum value that can be set with one scanning control parameter among the plurality of scanning control parameters in the ultrasonic diagnostic apparatus A.

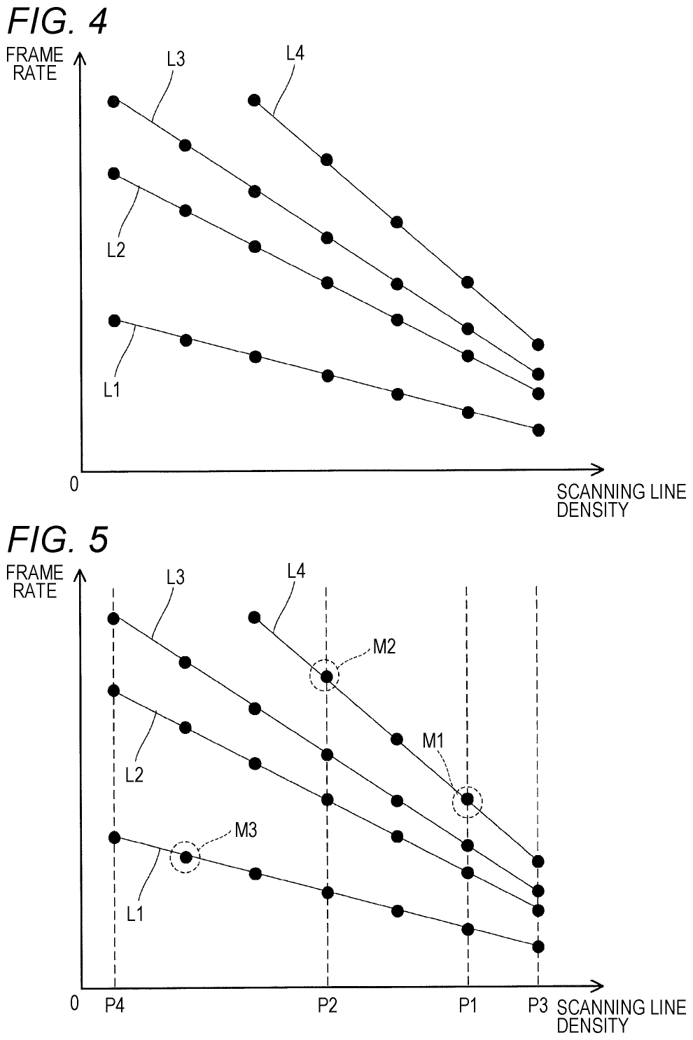

It is generally known that the plurality of scanning control parameters are in a so-called trade-off relationship. In the ultrasonic diagnostic apparatus A according to an embodiment of the present embodiment, for example, it has been experimentally confirmed that there is a mutual relationship as illustrated in FIG. 4. In FIG. 4, a vertical axis represents a frame rate, and a horizontal axis represents a scanning line density.

A solid line L1 is a scanning range corresponding to a maximum range (100%) of a scanning range of the ultrasonic diagnostic apparatus A. A solid line L2 is a scanning range corresponding to 50% of the maximum range of the scanning range of the ultrasonic diagnostic apparatus A. A solid line L3 is a scanning range corresponding to 40% of the maximum range of the scanning range of the ultrasonic diagnostic apparatus A. A solid line L4 is a scanning range corresponding to 30% of the maximum range of the scanning range of the ultrasonic diagnostic apparatus A.

Note that, in the following description, the scanning range is described as a width of the diagnostic region R, but the scanning range may be a view depth or both the width of the diagnostic region R and the depth of view.

Each scanning control parameter has a relationship in which, as one scanning control parameter is increased, another scanning control parameter decreases. For example, the frame rate decreases as the scanning line density is increased. Furthermore, the frame rate decreases as the scanning range is widened. In addition, as the frame rate is increased, the scanning line density becomes smaller and the scanning range becomes narrower.

Note that each of plots of the solid lines L1 to L4 in FIG. 4 indicates a value applicable to set values of the scanning line density and the frame rate. For example, when the scanning range is the maximum range, the values of the frame rate and the scanning line density corresponding to any plot on the solid line L1 are applied as the set values.

For example, it is assumed that one scanning control parameter among the plurality of scanning control parameters is the scanning line density, and a user performs operation of setting a range from an upper limit value P1 to a lower limit value P2 as illustrated in FIG. 5. The upper limit value P1 is a value smaller than a maximum value P3 that can be set in the ultrasonic diagnostic apparatus A, and the lower limit value P2 is a value larger than a minimum value P4 that can be set in the ultrasonic diagnostic apparatus A.

In this case, the range setting part 43 sets the setting range of the scanning line density to a range from the upper limit value P1 to the lower limit value P2.

Then, the determination part 42 determines a set value of the plurality of scanning control parameters on the basis of the setting range that is set by the range setting part 43. The determination part 42 determines values of the frame rate and the scanning range (scanning control parameters other than one scanning control parameter) such that the scanning line density (one scanning control parameter) becomes a value within the setting range.

For example, when increasing a resolution of an ultrasonic image, it is necessary to increase the scanning line density. In this case, the determination part 42 determines the scanning line density to be the upper limit value P1. Here, for example, when it is desired to increase the frame rate as well, a value (a plot surrounded by a circle dotted line M1) relating to the upper limit value P1 on the solid line L4 having a highest frame rate and a narrowest scanning range is determined as the set value of the scanning control parameter. Note that, in consideration of a balance between the scanning range and the frame rate, the set value of the scanning control parameter can be appropriately selected from the values relating to the upper limit value P1.

Furthermore, for example, when increasing the frame rate, it is necessary to lower the scanning line density and to narrow the scanning range. In this case, the determination part 42 determines the scanning line density to be the lower limit value P2, and determines a value on the solid line L4 (a plot surrounded by a circle dotted line M2) having a highest value of the frame rate, as the set value of the scanning control parameter. Note that, in consideration of the scanning range, the set value of the scanning control parameter can be appropriately selected from the values relating to the lower limit value P2.

As a result, values of the frame rate and the scanning range can be determined within the range of the scanning line density set to a range having a margin to some extent, so that the set value of the scanning control parameter can be flexibly set according to the purpose.

Note that the determination as to whether or not to increase any one of the resolution of the ultrasonic image and the frame rate can be appropriately determined according to a use by the user, a diagnosis purpose of the subject, a specification of the ultrasonic diagnostic apparatus A, or the like.

Furthermore, the determination part 42 may determine the plurality of scanning control parameters on the basis of a region of interest set by the region-of-interest setting part 41.

Since the region of interest is a portion that is subjected to enlargement display processing for the user to observe in detail, it is desirable to increase the resolution of the ultrasonic image. Therefore, for example, in a case where default values of a plurality of scanning control parameters are plots surrounded by a circle dotted line M3, it is necessary to determine the set value of the scanning control parameter so that the resolution of the ultrasonic image is higher than that of the plots. In the circle dotted line M3, the scanning range is the maximum range, and the scanning line density and the frame rate are relatively low values.

However, since the region of interest is a region set by the user, for example, in a case where the region of interest is set at a position not corresponding to a range (for example, as indicated by the solid line L2, 50% of the maximum range) in which the scanning range is narrowed, the scanning range has to be expanded.

Therefore, when the region of interest is set, the determination part 42 determines a plurality of scanning control parameters so that the resolution of the ultrasonic image increases, while considering the scanning range. For example, when the region of interest is set at a position corresponding to 30% of the maximum range of the scanning range, the determination part 42 sets a value of any plot (for example, a plot corresponding to the upper limit value P1 of the scanning line density) within a setting range on the solid line L4, as the set value of the scanning control parameter.

Further, when the region of interest is set at a position where the maximum range alone of the scanning range is applicable, among plots within the setting range on the solid line L1, the determination part 42 sets a value of a plot at which the scanning line density becomes the upper limit value P1, as the set value of the scanning control parameter.

In this way, it is possible to flexibly determine a set value of the scanning control parameter in consideration of a position of the region of interest.

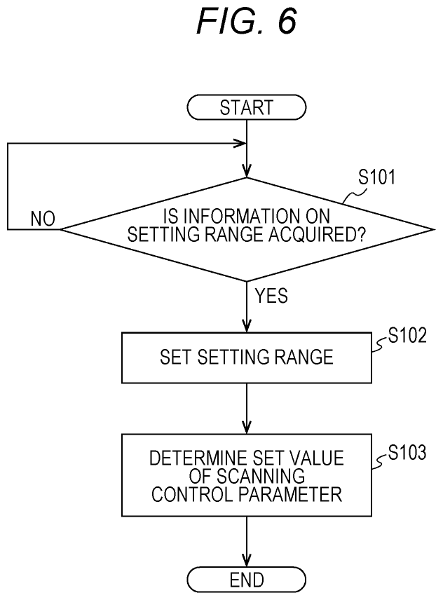

Next, an operation example when determination control on the scanning control parameter in the control part 40 is executed will be described. FIG. 6 is a flowchart illustrating an example of an operation example of determination control on a scanning control parameter in the control part 40. The processing in FIG. 6 is appropriately executed, for example, during diagnosis in the ultrasonic diagnostic apparatus A.

As illustrated in FIG. 6, the control part 40 determines whether or not information on a setting range of one scanning control parameter among a plurality of scanning control parameters has been acquired (step S101). As a result of the determination, when the information on the setting range has not been acquired (step S101, NO), the processing of step S101 is repeated.

Whereas, when the information on the setting range is acquired (YES in step S101), the control part 40 sets the setting range of the one scanning control parameter (step S102). Then, the control part 40 determines a set value of the plurality of scanning control parameters (step S103). Thereafter, this control ends.

According to the present embodiment configured as described above, a setting range of one scanning control parameter among a plurality of scanning control parameters is set, and a set value of the scanning control parameter is determined within the setting range. This makes it easy to determine, as the set value, a well-balanced value among the plurality of scanning control parameters having a mutual relationship.

For example, when one scanning control parameter is set to a fixed value, remaining scanning control parameters are inevitably limited. For example, in a case where the scanning line density is fixed to the upper limit value P1 in FIG. 5, a maximum value at which the frame rate can be determined is to be a value on the solid line L4, and the set value of the scanning control parameter is unable to be flexibly determined.

Whereas, in the present embodiment, the scanning line density can be variably set within a setting range from the upper limit value P1 to the lower limit value P2, so that the frame rate and the scanning range can be easily determined to well-balanced values within the setting range.

That is, in the present embodiment, since a range of selection of the set value among the plurality of scanning control parameters can be widened, the scanning control parameter can be flexibly set.

Note that, in the above embodiment, the range setting part 43 sets a setting range of one scanning control parameter among the plurality of scanning control parameters. However, the present invention is not limited thereto, and setting ranges of two or more scanning control parameters among the plurality of scanning control parameters may be set.

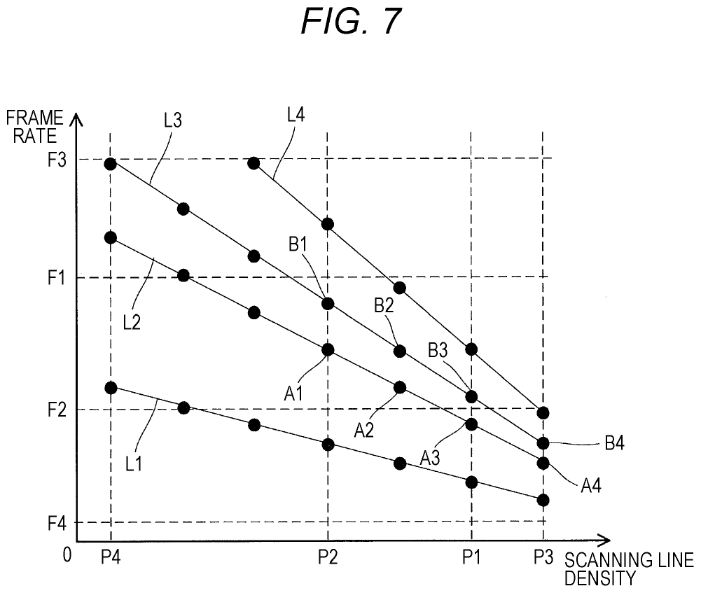

For example, it is assumed that two scanning control parameters among the plurality of scanning control parameters are the scanning line density and the frame rate. Then, as illustrated in FIG. 7, it is assumed that the user performs an operation of setting the scanning line density to a range from the upper limit value P1 to the lower limit value P2 and setting the frame rate to a range from an upper limit value F1 to a lower limit value F2.

The upper limit value P1 and the lower limit value P2 of the scanning line density are similar to those in the above embodiment. The upper limit value F1 of the frame rate is a value smaller than a maximum value F3 that can be set in the ultrasonic diagnostic apparatus A, and the lower limit value F2 is a value larger than a minimum value F4 that can be set in the ultrasonic diagnostic apparatus A.

In this way, a range of the set value of the scanning control parameter determined by the determination part 42 can be easily narrowed.

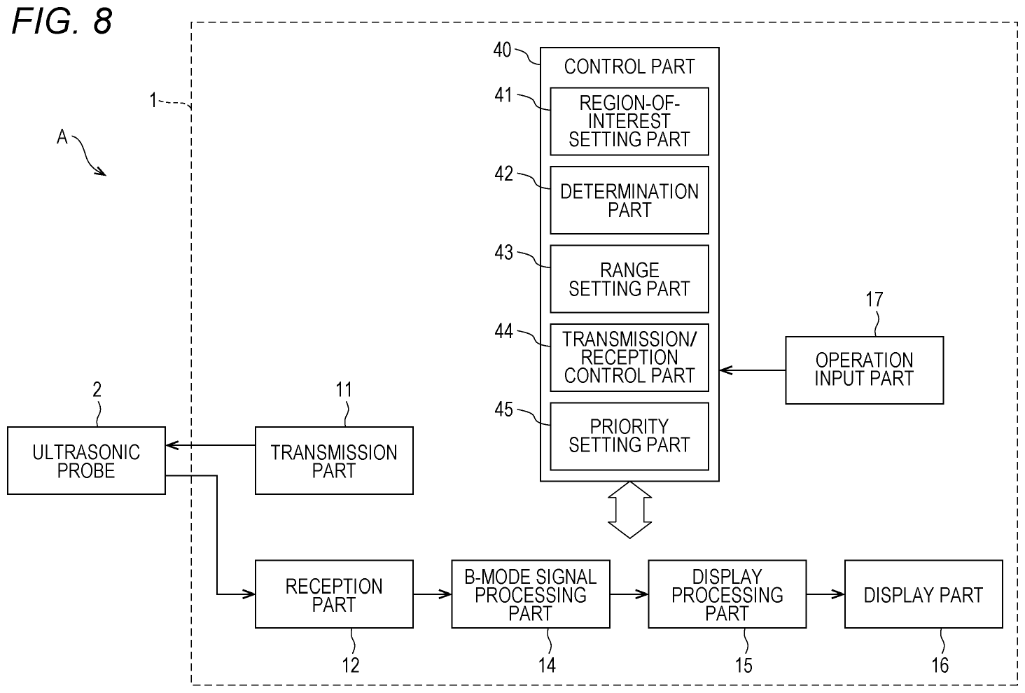

Furthermore, as illustrated in FIG. 8, the control part 40 may include a priority setting part 45. The priority setting part 45 sets a priority to upper limit values and lower limit values of individual setting ranges of two or more scanning control parameters. The determination part 42 determines a set value of a plurality of scanning control parameters in accordance with the priority.

The priority is, for example, a priority order given to upper limit values and lower limit values of individual setting ranges of two or more scanning control parameters, as a reference for determining a set value of the determination part 42.

For example, it is assumed that the priority setting part 45 sets the priority in the order of the lower limit value P2 of the scanning line density, the lower limit value F2 of the frame rate, the upper limit value P1 of the scanning line density, and the upper limit value F1 of the frame rate. A priority setting criterion of the priority setting part 45 may be any criterion such as one based on a user's instruction or one based on a specification of the ultrasonic diagnostic apparatus A.

For example, in FIG. 7, in a case where the scanning range is 50% (the solid line L2), since the highest priority is the lower limit value P2 of the scanning line density, A1, A2, A3, and A4, which are four plots equal to or greater than the lower limit value P2, are to be candidates for the set value of the scanning control parameter.

A1 is a plot corresponding to a value of a lowest scanning line density and a highest frame rate among the four plots. A2 is a plot corresponding to a value of a second lowest scanning line density and a second highest frame rate among the four plots. A3 is a plot corresponding to a value of a second highest scanning line density and a second lowest frame rate among the four plots. A4 is a plot corresponding to a value of a highest scanning line density and a lowest frame rate among the four plots.

While the second highest priority is the lower limit value F2 of the frame rate, among the above four plots, A1 and A2 are equal to or greater than the lower limit value F2 of the frame rate, and thus these two are to be candidates for the set value of the scanning control parameter.

Since the third highest priority is the upper limit value P1 of the scanning line density, A2 that is equal to or less than the upper limit value P1 and closest to the upper limit value P1 among these two is determined as the set value of the scanning control parameter.

Furthermore, for example, in a case where the scanning range is 40% (the solid line L3), since the highest priority is the lower limit value P2 of the scanning line density, B1, B2, B3, and B4, which are four plots equal to or greater than the lower limit value P2, are to be candidates for the set value of the scanning control parameter.

B1 is a plot corresponding to a value of a lowest scanning line density and a highest frame rate among the four plots. B2 is a plot corresponding to a value of a second lowest scanning line density and a second highest frame rate among the four plots. B3 is a plot corresponding to a value of a second highest scanning line density and a second lowest frame rate among the four plots. B4 is a plot corresponding to a value of a highest scanning line density and a lowest frame rate among the four plots.

While the second highest priority is the lower limit value F2 of the frame rate, among the above four plots, B1, B2 and B3 are equal to or greater than the lower limit value F2 of the frame rate, and thus these three are to be candidates for the set value of the scanning control parameter.

Since the third highest priority is the upper limit value P1 of the scanning line density, B3 that is equal to or less than the upper limit value P1 and closest to the upper limit value P1 among these two is determined as the set value of the scanning control parameter.

By setting the priority in this manner, the set value of the scanning control parameter by the determination part 42 can be easily determined automatically.

Furthermore, the priority is not limited to the above-described order, and can be appropriately set. For example, it is assumed that the priority is set in the order of the lower limit value P2 of the scanning line density, the lower limit value F2 of the frame rate, the upper limit value F1 of the frame rate, and the upper limit value P1 of the scanning line density. That is, it is assumed that the third highest priority and the lowest priority among the priorities in FIG. 7 are switched.

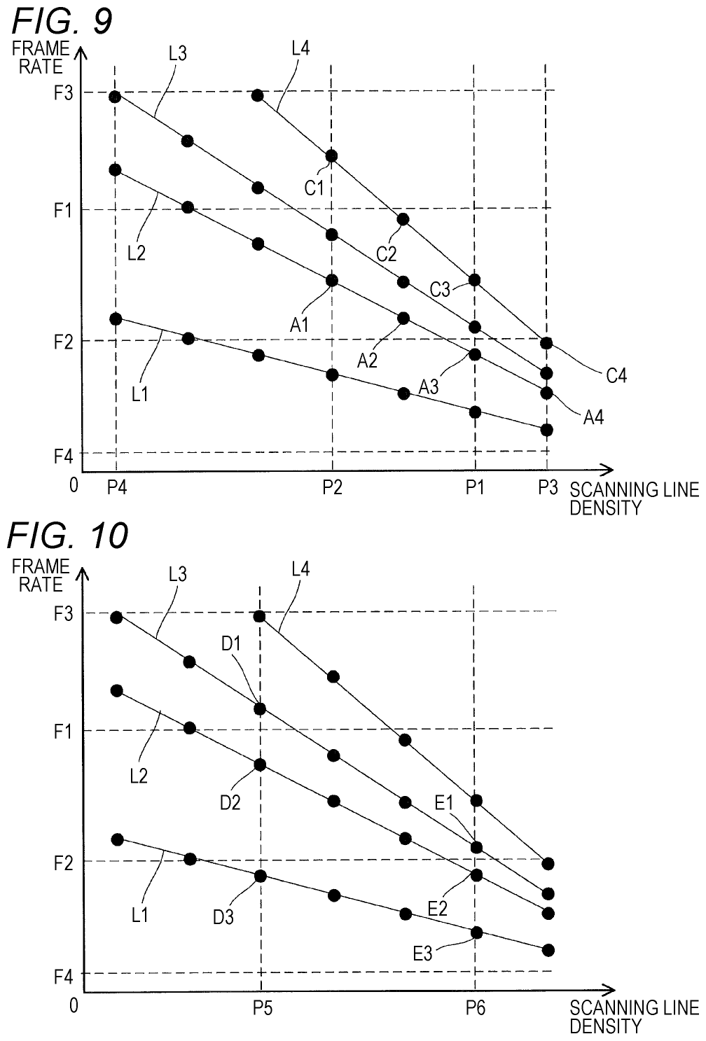

For example, as illustrated in FIG. 9, in a case where the scanning range is 50% (the solid line L2), since the highest priority is the lower limit value P2 of the scanning line density, A1, A2, A3, and A4, which are four plots equal to or greater than the lower limit value P2, are to be candidates for the set value of the scanning control parameter. A1, A2, A3, and A4 are similar to A1, A2, A3, and A4 illustrated in FIG. 7.

While the second highest priority is the lower limit value F2 of the frame rate, among the above four plots, A1 and A2 are equal to or greater than the lower limit value F2 of the frame rate, and thus these two are to be candidates for the set value of the scanning control parameter.

Since the third highest priority is the upper limit value F1 of the frame rate, A1 closest to the upper limit value F1 among these two is determined as the set value of the scanning control parameter.

Furthermore, in a case where the scanning range is 30% (the solid line L4), since the highest priority is the lower limit value P2 of the scanning line density, C1, C2, C3, and C4, which are four plots equal to or greater than the lower limit value P2, are to be candidates for the set value of the scanning control parameter.

C1 is a plot corresponding to a value of a lowest scanning line density and a highest frame rate among the four plots. C2 is a plot corresponding to a value of a second lowest scanning line density and a second highest frame rate among the four plots. C3 is a plot corresponding to a value of a second highest scanning line density and a second lowest frame rate among the four plots. C4 is a plot corresponding to a value of a highest scanning line density and a lowest frame rate among the four plots.

While the second highest priority is the lower limit value F2 of the frame rate, among the above four plots, C1, C2 and C3 are equal to or greater than the lower limit value F2 of the frame rate, and thus these three are to be candidates for the set value of the scanning control parameter.

Since the third highest priority is the upper limit value F1 of the frame rate, C2 that is equal to or less than the upper limit value F1 and closest to the upper limit value F1 among these three is determined as the set value of the scanning control parameter.

Further, in FIGS. 7 and 9 described above, an example in which two parameters of the plurality of scanning control parameters are the scanning line density and the frame rate has been described. However, the present invention is not limited thereto, and for example, the two parameters may be a combination other than the scanning line density and the frame rate.

For example, it is assumed that two parameters among the plurality of scanning control parameters are the scanning range and the frame rate. Then, as illustrated in FIG. 10, it is assumed that the user performs an operation of setting the scanning range to a range of an upper limit value of L2 (50%) and a lower limit value of L3 (40%) and setting the frame rate to a range of the upper limit value F1 to the lower limit value F2.

Here, for example, it is assumed that the priority setting part 45 sets the priority in the order of L3 that is a lower limit value of the scanning range, F2 that is a lower limit value of the frame rate, F1 that is an upper limit value of the frame rate, and L2 that is an upper limit value of the scanning range.

For example, in FIG. 10, in a case where the scanning line density is P5, since the highest priority is L3 that is the lower limit value of scanning range, D1, D2, and D3 that are three plots to be equal to or greater than L3 are to be candidates for the set value of the scanning control parameter. P5 is a value of the scanning line density corresponding to the third smallest plot in L1, for example.

D1 is a plot corresponding to a value of a narrowest scanning range and a highest frame rate among the three plots. D2 is a plot corresponding to a middle value of the scanning range and a middle value of the frame rate among the three plots. D3 is a plot corresponding to a value of a widest scanning range and a lowest frame rate among the three plots.

Since the second highest priority is the lower limit value F2 of the frame rate, D1 and D2 that are equal to or greater than the lower limit value F2 of the frame rate among the above three plots are to be candidates for the set value of the scanning control parameter.

While the third highest priority is the upper limit value F1 value of the frame rate, D2 is equal to or less than the upper limit value F1 of the frame rate among the above two plots, and thus D2 is determined as the set value of the scanning control parameter.

Furthermore, for example, in a case where the scanning line density is P6, since the highest priority is L3 that is the lower limit value of scanning range, E1, E2, and E3 that are three plots to be equal to or greater than L3 are to be candidates for the set value of the scanning control parameter. P6 is a value of the scanning line density corresponding to the second largest plot in L1, for example.

E1 is a plot corresponding to a value of a narrowest scanning range and a highest frame rate among the three plots. E2 is a plot corresponding to a middle value of the scanning range and a middle value of the frame rate among the three plots. E3 is a plot corresponding to a value of a widest scanning range and a lowest frame rate among the three plots.

While the second highest priority is the lower limit value F2 of the frame rate, E1 is equal to or greater than the lower limit value F2 of the frame rate among the above three plots, and thus E1 is determined as the set value of the scanning control parameter.

In addition, the priority setting part 45 sets the priority to four among upper limit values and lower limit values of individual setting ranges of two or more scanning control parameters. However, the present invention is not limited thereto, and it is sufficient that the priority is set to at least two among upper limit values and lower limit values of individual setting ranges. For example, the priority setting part 45 may set the priority of a combination of individual upper limit values alone or may set the priority of a combination of individual lower limit values alone. Further, the priority setting part 45 may set a priority of a combination of an upper limit value of a setting range of one scanning control parameter and a lower limit value of another scanning control parameter.

Further, in the above embodiment, the upper limit value and the lower limit value of the setting range are absolute values of the scanning control parameter. However, the present invention is not limited thereto, and the upper limit value and the lower limit value may be relative values of the scanning control parameter with respect to a value of a predetermined reference state.

The predetermined reference state is a state before the setting range is set by the range setting part 43, and is, for example, a state corresponding to a default value, a state in which an ultrasonic image is subjected to non-enlargement display processing by the display processing part 15, or the like.

By using this relative value, when the scanning control parameter is determined by the determination part 42, the user can easily grasp how the scanning control parameter varies with respect to the reference state.

Further, in the above embodiment, the region-of-interest setting part 41 is provided. However, the present invention is not limited thereto, and the region-of-interest setting part may not be provided.

In addition, the above-described embodiment is merely an example of implementation in carrying out the present invention, and the technical scope of the present invention should not be construed in a limited manner by these. That is, the present invention can be implemented in various forms without departing from the scope or main features of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic diagnostic apparatus for generating an ultrasonic image of a subject in accordance with a set value of a plurality of scanning control parameters having a mutual relationship, the ultrasonic diagnostic apparatus comprising:
a hardware processor that
sets a setting range narrower than a limit range of the ultrasonic diagnostic apparatus, for at least one scanning control parameter among the plurality of scanning control parameters; and
determines a set value of the plurality of scanning control parameters based on the setting range.

2. The ultrasonic diagnostic apparatus according to claim 1, wherein
the hardware processor determines a value of a scanning control parameter other than the at least one scanning control parameter, to cause the at least one scanning control parameter to have a value within the setting range.

3. The ultrasonic diagnostic apparatus according to claim 1, wherein
the hardware processor is capable of setting a setting range of two or more scanning control parameters among the plurality of scanning control parameters.

4. The ultrasonic diagnostic apparatus according to claim 3, wherein
the hardware processor sets a priority to at least two among upper limit values and lower limit values of individual setting ranges of the two or more scanning control parameters, and
determines a set value of the plurality of scanning control parameters based on the priority.

5. The ultrasonic diagnostic apparatus according to claim 1, wherein
the hardware processor sets the setting range based on an instruction of a user.

6. The ultrasonic diagnostic apparatus according to claim 1, wherein
the plurality of scanning control parameters include a scanning line density, a scanning range, and a frame rate.

7. The ultrasonic diagnostic apparatus according to claim 1, further comprising:
a display processing part that performs display processing on the ultrasonic image; wherein
the hardware processor sets a region of interest of the ultrasonic image, and
the display processing part performs enlargement display processing on the region of interest.

8. The ultrasonic diagnostic apparatus according to claim 7, wherein
the hardware processor determines a set value of the plurality of scanning control parameters based on the region of interest.

9. The ultrasonic diagnostic apparatus according to claim 1, wherein
an upper limit value and a lower limit value of the setting range are absolute values of each of the scanning control parameters.

10. The ultrasonic diagnostic apparatus according to claim 1, wherein
an upper limit value and a lower limit value of the setting range are relative values with respect to a value of a predetermined reference state in each of the scanning control parameters.

11. The ultrasonic diagnostic apparatus according to claim 10, wherein
the predetermined reference state is a state before the setting range is set by the hardware processor.

12. A determination method for a set value of a plurality of scanning control parameters of an ultrasonic diagnostic apparatus for generating an ultrasonic image of a subject in accordance with a set value of the plurality of scanning control parameters having a mutual relationship, the determination method comprising:
setting a setting range narrower than a limit range of the ultrasonic diagnostic apparatus, for at least one scanning control parameter among the plurality of scanning control parameters; and
determining a set value of the plurality of scanning control parameters based on the setting range.

13. A non-transitory recording medium storing a computer readable determination program for a set value of a plurality of scanning control parameters of an ultrasonic diagnostic apparatus for generating an ultrasonic image of a subject in accordance with a set value of the plurality of scanning control parameters having a mutual relationship,
the determination program causing a computer to execute:
setting a setting range narrower than a limit range of the ultrasonic diagnostic apparatus, for at least one scanning control parameter among the plurality of scanning control parameters; and
determining a set value of the plurality of scanning control parameters based on the setting range.
